(19) United States
(12) Patent Application Publication
(10) Pub. No.: US 2024/0123637 A1
(43) Pub. Date: Apr. 18, 2024

(54) UTILITARIAN TASK-BASED CONTAINER AND INFLATABLE ISOLATION CHAMBER FOR MEDICAL, MILITARY AND TRAINING APPLICATIONS

(71) Applicants: Mike Horia Mihail TEODORESCU, Cambridge, MA (US); SURGIBOX INC., Cambridge, MA (US)

(72) Inventor: Mike Horia Mihail Teodorescu, Cambridge, MA (US)

(73) Assignee: SURGIBOX INC., Cambridge, MA (US)

(21) Appl. No.: 18/277,230

(22) PCT Filed: Feb. 20, 2022
(86) PCT No.: PCT/US2022/017117

§ 371 Date: Aug. 15, 2023

Related U.S. Application Data
(63) Continuation-in-part of application No. PCT/US2021/058496, filed on Nov. 8, 2021.
(60) Provisional application No. 63/154,761, filed on Feb. 28, 2021.
(60) Provisional application No. 63/247,545, filed on Sep. 23, 2021.

(51) Int. Cl. B25J 21/02

(52) U.S. Cl. CPC B25J 21/02; A61B 90/40

(57) ABSTRACT

The invention discloses inflatable-containers, also named utilitarian task-based containers, that essentially include a set of prepacked materials, ingredients, devices, tools, and/or instruments which could be used for performing a group of related actions, such as: preparing medical materials and equipment which could be used during an in-the-field medical interventions and surgeries performed via portable surgical systems. The containers may include inward and/or outward protuberances shaped as gloves with sleeves, fingers, and handlers to allow manipulations as necessary for the predefined task. The inflatable-containers may be transparent so that an user can see within the interior of the container. The containers may be designed to for single use or for repeated uses. The inflatable-containers may be self-inflating.

### CROSS REFERENCE TO RELATED APPLICATIONS

This application claims priority from and the benefit of the U.S. Provisional Patent Application No. 63/154,761 filed on Feb. 28, 2021 and titled “UTILITARIAN TASK-BASED CONTAINER AND INFLATABLE ISOLATION CHAMBER”, the U.S. Provisional Patent Application No. 63/247,545 filed on Sep. 23, 2021 and titled “PORTABLE SYSTEM FOR ISOLATION AND REGULATION OF SURGICAL SITE ENVIRONMENTS”, and the International Patent Application no. PCT/US2021/058496 filed on Nov. 8 2021 titled “PORTABLE SYSTEM FOR ISOLATION AND REGULATION OF SURGICAL SITE ENVIRONMENTS” which are hereby incorporated by reference for all purposes as if fully set forth herein.

### BACKGROUND OF THE INVENTION

### I. Field of the Invention

Exemplary embodiments of the present invention relate to utilitarian task based containers for regulating medical, military, laboratory, training and other environments.

### II. Discussion of the Background

There is a need for portable medical equipment enabling in-the-field medical interventions such as in remote regions (e.g., mountains, deserts, and remote islands) where there is no quick and proper access to medical facilities.

Over 25% of the global disease burden requires surgical therapy, which could prevent over 18 million deaths per year. These range from obstetric complications to traumas to infections to cancer and beyond. Yet 2 billion people have no meaningful access to safe surgical care, and 2-3 billion more have access only to unsterile surgeries in contaminated environments, leading to disproportionate rates of surgical infections. Innovations in this field typically focus upon making operating rooms and operating room ventilation systems more mobile, such as in tent format. However, such systems remain costly to purchase and to maintain. Moreover, such systems are difficult to transport rapidly to remote areas. At the same time, over 85,000 medical providers are infected by patient bodily fluids annually, with 90% of infected providers worldwide having been exposed while working in low-resource settings. While personal protective equipment mitigates these risks to some extent, there is a definite trade-off between the level of protection and both the cost as well as the user comfort, which is well-documented to correspond to user compliance.

The following patent applications are addressing some of the above challenges: PCT international patent application no. PCT/US2017/04226 titled “Ultraportable system for intraoperative isolative and regulation of surgical site environments”; PCT international patent application no. PCT/US2019/032148 titled “Sterile sleeves for portable surgical systems”; PCT international patent application no. PCT/US2020/032280 titled “Systems and methods for intraoperative isolation and control of surgical site environments”; and PCT international patent application no. PCT/US2019/051502 titled “Data analytics and interface platform for portable surgical enclosure”. The above patent applications describe portable systems, devices and methods for regulating surgical site environment addressing some of the challenges of patient and provider intraoperative exposure to infectious risks and airborne particulates by implementing ultraportable, self-contained, passive and active, bilateral barriers against exchange of contaminants between incisions and the greater surgical area. The above patent applications are incorporated hereinafter as if fully set forth herein.

There is a need for enclosures or containers in which medical operators (e.g., in-the-field operators) store, access and prepare medical tools, materials, implants, equipment in an efficient, sterile and safe way (both for the patient and for the operator). In particular, there is a need for utilitarian containers that can be used by medical operators in conjunction with the portable systems, devices and methods for regulating surgical site environments described in the above patent applications.

There is also a need for portable enclosures and containers that could help military personnel prepare and operate materials, equipment, and tools in a safe and/or sterile way while in various environments, such as in-the-field missions.

In addition, there are many needs associated with training of personnel (e.g., military personnel, medical personnel, various professionals performing in-the-field tasks), education of students, and in all educational environments (e.g., primary schools, high school, college). Training and educational experiments are narrow tasks with the purpose of learning and skill development. These experiments are essential for creating skills and knowledge of military, medical, and other personnel as well as of students (starting from elementary school to college). For example, many educational experiments that are hazardous or produce inconvenient smells and fumes cannot be performed because schools and colleges do not have at all or have an insufficient number of the required fume hoods and gloveboxes, gloves, protecting glasses and glass bels, and other equipment for safe experimentation. In addition, several experiments are not performed by lower grade students because of safety concerns. Some educational experiments could benefit younger students and even preschool children but cannot be performed by them because the experiments may be hazardous for inexperienced students without basic skills of using protective means and procedures.

The inventions in this application provide solutions to the above needs in the education field, such as experiments prepacked in airtight, chemically inert, and electrically isolated containers which can be performed by manipulation, from the exterior of the container, of the materials, biological materials, devices, tools, and instruments used in the experiment and included in the container. The inventions in this application also provide solutions in the military and medical fields to the above needs, such as test-kits/operation-kits prepacked in airtight, chemically inert, and electrically isolated containers which can be performed by manipulation, from the exterior of the container, of the materials, biological materials, devices, tools, and instruments used in the experiment and included in the container. The inventions herein may help a large number of trainees and students to grasp scientific and technological knowledge and skills that otherwise may be attained only in higher grades, or only at college or university level.

The above information disclosed in this Background section is only for enhancement of understanding of the background of the invention and therefore it may contain information that does not form any part of the prior art.

### SUMMARY OF THE INVENTION

Exemplary embodiments of the present invention provide inflatable utilitarian-containers (also referred as utilitarian task-based containers) which may include a set of prepacked materials, ingredients, devices, tools, and/or instruments that could be used to perform a group of related actions, such as: preparing medical materials and/or equipment which could be used during in-the-field medical interventions and surgeries performed via portable surgical systems; preparing military equipment and/or materials which could be used during in-the-field missions and operations; preparing materials and/or equipment which could be used for in-the-field testing by various professionals (e.g., agriculture, electricians, technicians, civil engineers, etc.). The utilitarian-containers may include inward and/or outward protuberances shaped as gloves with sleeves, fingers, and handlers to allow manipulations as necessary for the predefined task. The utilitarian-containers may be transparent so that an user can see within the interior of the container. The containers may be designed for single use or for repeated uses. The utilitarian-containers may be self-inflating.

Exemplary embodiments of the invention provide generally prepacked sets which may include utilitarian-containers enabling personnel to perform specific tasks in a safe and contained environment. The tasks may involve performing experiments; performing measurements of potentially hazardous materials; performing medical tasks such as preparing medical tools and equipment to be used in surgical procedures; performing medical tests including pathology, blood tests, sensitive cultures, or a combination thereof.

Exemplary embodiments of the invention may provide generally prepacked sets including an utilitarian-container enabling military personnel to perform in-the-filed specific tasks in a safe and sterile environment. The tasks may include one or more of the following: preparing tools, equipment and/or materials for military operations; performing tests; performing experiments; etc.

Exemplary embodiments of the invention may provide a low cost, lightweight, fully portable, self-inflatable container providing a safely enclosed environment for performing specific tasks (such as preparation of equipment, tools and materials, performing experiments, performing tests, etc.). The container walls may act as a safety barrier for the user, while allowing all manipulations as needed for the task. The manipulations may be performed by the user or users from outside the container. The container may include materials, devices, tools, implements, and instruments required by the planned task. The container may carry outside it, on its walls, other devices useful for the task.

Exemplary embodiments of the invention also disclose containers which may include a set of prepacked materials, ingredients, devices, tools, instruments, toys, and/or game parts for performing a group of related actions constituting a specific task, such as: an educational experiment, a food preparation task, or an educational game. The prepacked materials, ingredients, devices, tools, instruments, toys, and/or game parts may be included in a safe, inflatable or self-inflating, transparent container for single or repeated uses, wherein the container may include inward and outward protuberances shaped as gloves with sleeves, fingers, and handlers to allow manipulations as necessary for the predefined task.

Exemplary embodiments of the invention may provide prepacked sets configured to perform specified tasks, such as educational experiments. The prepacked sets may include toy-like experimental kits. The prepacked sets may include a safe, transparent, inflatable plastic containers, preferably provided with means for self-inflation, wherein the inflatable containers include equipment and materials which may be used for performing measurements, experiments, test, material preparation. The invention proposes and materializes notions such as “laboratory in a bubble”, “kitchen in a bubble” and “bubbled games”, with the tasks ranging from cooking to experimenting in disciplines comprising chemistry, physics, biology, and geo-morphology.

The invention may provide a low cost, lightweight, fully portable, self-inflatable container configured to create a safely enclosed environment for performing specific tasks, such as educational experiments or meal preparation. The container includes an enclosure having walls acting as a safety barrier for the user, while allowing all manipulations as needed for the task. The manipulations may be performed by the user or users from outside the container. The container may include materials, devices, tools, implements, and instruments required by the planned task. The container may carry outside it, on its walls, other devices useful for the task.

The inflatable containers and the included tools, objects, kits, and materials may include and may be connected to sensors and communication means for the collection and further processing of the collected data during tasks and procedures.

The utilitarian task-based container may be designed to fit specific, typically narrow purpose such as medical, military, educational, demonstrative, or meal preparation purpose, with a specific type of user in mind, with the container and the content synergistically related, with the dimensions and shape of the inflated container. The properties of the container and its walls may be optimized to the tasks/procedures to be performed, to the quantities of materials and samples required by the tasks, to the devices and tools needed for accomplishing the task, and to the intended type of user. The materials, samples, devices and tools included in the container may be essentially determined by the characteristics of the task and the intended users, may be adapted to the purpose of the task and to the users of the container, and may be optimized for the task.

The tools included in the set may be dimensionally optimized to the quantities of materials and to the dimensions of the devices to be manipulated during the task accomplishment. The tools included in the set may be dimensionally optimized to the intended users, with the dimensions reduced and tool manipulation simplified when the intended users are children such as for an educational experiment kit. The utilitarian task-based container may be optimized for different age groups of users. The positions of the tools and sleeves may be optimally determined for the height and shoulder width of the intended user.

For example, an embodiment of the invention may provide an experiment container configured to be used for educational purposes, wherein the experiment container may be designed to fit users of ages 10-12 and may have a different sleeve and tool positioning with respect to the wall of the container than an experiment container designed for users of ages 15-18, so as to be of the optimal height and width for different age groups of students. Another embodiment of the invention may provide an utilitarian task-based container designed for cooking inside the container, wherein the container may have optimizations in tool placement and sleeve positioning to increase ease of use for elderly users.

Educational experiments may be performed inside a sealed task-based utilitarian container which may include a viewing window for students and teachers to observe the experiment or in a task-based utilitarian container including transparent walls (in part or fully transparent).

The task-based utilitarian container may include or may come equipped with tools needed to manipulate the elements of the experiment (steps of the task) and may be sealed from the outside environment. The task-based utilitarian container may contain an atmosphere of inert gas, or, depending on the experiment, may be inflated with compressed air, CO2, or by other means known in the art. The task-based utilitarian container may include a one-way exhaust valve, which may be child-safe and/or which may be operated by a teacher or supervisor after the task is accomplished to vent the internal atmosphere of the task-based utilitarian container, for example in a fume hood. The task-based utilitarian container may be configured to be discarded after accomplishment of the task/procedure (e.g., an educational experiment, a test, an operation, a procedure). The task-based utilitarian container may be configured to be reusable.

The task-based utilitarian container may be used to perform experiments which are hazardous and especially hazardous for pre-school and lower grade school children, such as chemical experiments that use corrosive or toxic materials (e.g., stronger chemical bases, acids, light or heavy metals, noxious chemical compounds); experiments involving chemical reactions producing or involving toxic or corrosive compounds; experiments involving chemical reactions that can eject materials, especially hot or noxious powders, liquids or gases; chemical or physical experiments that use or produce micro- or nano-powders or fibers; experiments that involve high electric potentials or currents; experiments that require or produce heat that may be hazardous for children; experiments that involve or may free into the environment biological microorganisms or biological materials; and any combinations of the above experiments. In an exemplary embodiment, the utilitarian container may be used for experiments in outer space.

The task-based utilitarian container may be used in other circumstances and may mitigate other problems in addition to hazardous experiments. The task-based utilitarian container may be used for experiments and procedures generating inconvenient materials such as: gases/materials generating a bad smell; inconvenient liquids such as those that mildly corrode, strain, dye, or otherwise produce undesired effects, including allergies.

The solution of utilitarian containers for specified tasks, including experiments, according to the invention, may be unsuitable when large quantities of heat or gas are produced, that may result in burns through the walls of the container, to fires, leaks through the container or cracks in the walls with subsequent freeing of noxious materials in the environment.

It is to be understood that both the foregoing general description and the following detailed description are exemplary and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of this specification, illustrate embodiments of the invention, and together with the description serve to explain the principles of the invention.

FIG. 1 shows a view of an embodiment of the utilitarian task-based container.

FIG. 2 shows a section in the wall of the utilitarian task-based container including two fingers, a tool permanently attached to the fingers on the inner side, and a section through the hand using the tool.

FIG. 3 shows an embodiment of the utilitarian task-based container comprising inflatable beams and/or girds.

### DETAILED DESCRIPTION

The invention is described more fully hereinafter with reference to the accompanying drawings, in which embodiments of the invention are shown. This invention may, however, be embodied in many different forms and should not be construed as limited to the embodiments set forth herein. Rather, these embodiments are provided so that this disclosure is thorough, and will fully convey the scope of the invention to those skilled in the art. In the drawings, the size and relative sizes of layers and regions may be exaggerated for clarity. Like reference numerals in the drawings denote like elements.

The following detailed description is provided to gain a comprehensive understanding of the methods, apparatuses and/or systems described herein. Various changes, modifications, and equivalents of the systems, apparatuses and/or methods described herein will suggest themselves to those of ordinary skill in the art. Descriptions of well-known functions and structures are omitted to enhance clarity and conciseness.

It will be understood that when an element or layer is referred to as being “on” or “connected to” another element or layer, it can be directly on or directly connected to the other element or layer, or intervening elements or layers may be present. In contrast, when an element or layer is referred to as being “directly on” or “directly connected to” another element or layer, there are no intervening elements or layers present. It will be understood that for the purposes of this disclosure, “at least one of X, Y, and Z” can be construed as X only, Y only, Z only, or any combination of two or more items X, Y, and Z (e.g., XYZ, XY, YY, YZ, ZZ).

Throughout the description and the claims, the terms container, utilitarian-container, and utilitarian task-based container are used as equivalent terms. The term “in-the-field operations” is used with the meaning of operations performed in areas which are far from medical facilities, military bases, hospitals. In-the-field operations include operations conducted in remote areas such as mountains, deserts and islands.

Exemplary embodiments of the invention provide utilitarian-containers including pre-packed kits (also referred as testing kit/set or experimental kit/set) configured to be used for tasks and applications such as: in-the-field medical/surgical interventions, in-the-field military applications, research, household activities, educational experiments, and games.

When the task is an experiment or test, the testing kit and/or experimental set is at least in part included in an enclosure of the utilitarian-container. The testing kits may include materials, biological samples, devices, instruments, tools, and labware as required by the specified experiment or experiments. Prepacked experiments can be used when large numbers of repeated tests are needed, such as the tests required by various biological and medical analyses. The experiment/tests may be performed during and/or in conjunction with medical interventions. The experiment/tests may be performed by military personnel during in-the-field operations.

The utilitarian-container may be used for educational experiments and games. The utilitarian-container is particularly useful and may be used for experiments that are meant for educational/recreational purposes through experimentation or experiment-like activities when the experiments may produce various hazards or inconveniences when performed by direct contact or in open atmosphere.

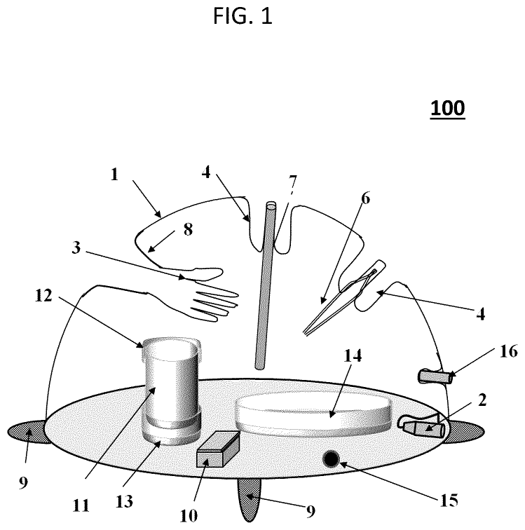

FIG. 1 shows a view of an embodiment of the utilitarian task-based utilitarian container 100 including an upper wall of semispherical shape, a glove with a sleeve, and two pairs of ‘fingers’ (glove-like inner protuberance for two fingers only) with tools attached. The utilitarian-container 100 may include an enclosure 1, which may be preferably provided with means for self-inflation such as gas cartridge 2. The walls of enclosure 1 may be transparent and inflatable. The enclosure 1 may be made of plastic. The enclosure 1 may be sealed and preserved sealed during storage, transportation and use. The enclosure walls may act as a protective barrier for the users and for the environment as well, and may preserve a controlled atmosphere inside the enclosure. The enclosure may substantially contain the entire set of materials, devices, labware, tools, and instruments needed for performing the task. Users may perform the necessary manipulations in the enclosure 1 using a set of gloves, fingers, tools, and protuberances formed in the walls of the enclosure. FIG. 1 also shows examples of containers for the materials used in the experiments, a compressed gas cartridge for inflation, an external camera, and fasteners.

Various means for self-inflation of enclosures and containers may be used as well known in the art. For example, US International Patent Application no. PCT/GB2015/051187 by inventor James Haliburton (titled “Self-inflating balloon”) teaches the inflation of balloons or other inflatable objects with gas from a compressed gas canister or cartridge. U.S. Pat. No. 5,049,106 by inventors Sunyong Kim and Dae W. Lee (titled “Self-contained, self-inflating novelty balloon”) teaches about a similar way of self-inflation.

In an exemplary embodiment of the invention the materials, kits, biological samples, devices, instruments, and tools needed in the performance of a task are included in an airtight enclosure that acts as a mechanical, electrical, chemical, and biological barrier between the inner medium where the task is performed and the external environment of the user. The inner space of the enclosure (which is also an inner space of the utilitarian-container) may be inflated preferably autonomously by means of a gas releasing device 2 pre-packed in the enclosure.

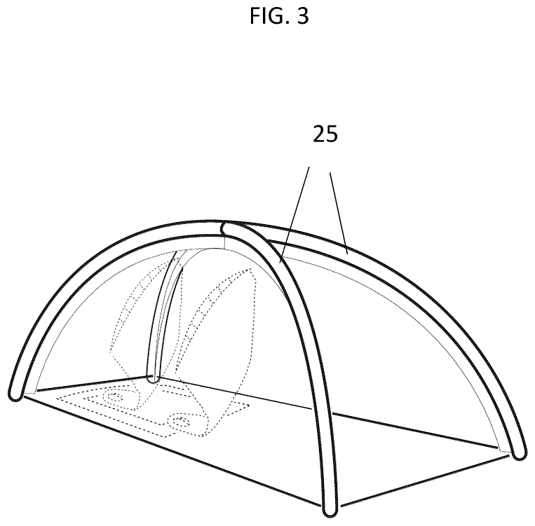

FIG. 3 shows an embodiment of the utilitarian task-based container comprising inflatable beams 25 and/or girds. When the required dimensions of the enclosure are large, the enclosure may be reinforced with self-inflating ribs and girds supporting the walls of the enclosure and positioned and connected to the said walls inside or outside the enclosure. The beams and girds may be inflated at significantly higher pressure than the enclosure.

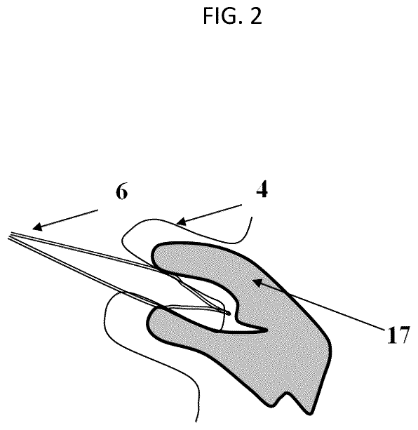

The enclosure may include inward protuberances formed as gloves 3 or fingers 4 to allow manipulation without directly contacting the elements inside the enclosure/utilitarian-container. In addition, the container may include means, using fasteners, for conveniently handling the container and attaching it during the use to other surrounding objects. Also, the fingers may include tools permanently attached to them, such as pincers 6 and stirring rods or spatulas 7. FIG. 2 shows a section in the wall of an utilitarian task-based container including two fingers 4, a tool 6 permanently attached to the fingers on the inner side, and a section through the hand 17 using the tool.

The shape, dimensions, number of fingers, gloves, and protuberances aimed to accommodate tools used from the exterior of the container or from the interior may be designed and configured to fit various circumstances, such as: the intended number of users using the container simultaneously, the age of the intended users, the processes involved by the task performance. The shape and dimensions of the gloves 3, glove sleeves 8, fingers, and other protuberances may be designed and configured to fit the circumstances of specific type of users, such as medical personnel, military personnel, elementary school students, adolescents, adults, and elderly people. Therefore, the fingers and gloves with their sleeves may be designed and configured to have dimensions suitable for the hands and fingers of the users and to have positions corresponding to the normal, ergonomically and convenient use by the targeted users and the task. For example, the distance between the right and left hand gloves may be smaller for children because the normal distance between their forearms during work is smaller than for adults and may be depending on the distances between their shoulders.

The positioning of the tools and protuberances in the utilitarian task-based container wall to manipulate said tools inside the environment of the utilitarian task-based container, as well as the number and positioning of gloves, may be configured to fit the specific task. The utilitarian task-based container may be configured to include all the necessary elements to the performance of the desired task. The tools, materials, and any other elements necessary for the performance of the task are specific to the task and may be secured to the inner walls of the utilitarian task-based container, including but not limited to permanently affixed into position.

The locations of the gloves 3, fingers 4 and protuberances on the container walls may be determined by the intended manipulations to be performed for the task attainment and by ergonomic considerations and consideration of the skills and abilities of the targeted type of user. Among others, the distance between the bases of the left-hand glove and the right-hand glove or between the respective arm sleeves may be approximately the distance between the hands or forearms of the operator during manipulations in free space, taking into account the age of the user.

The utilitarian-container may include one or more attachment/locking means 9 for maintaining its position over the working table, kitchen table, laboratory bench, or similar environments. These attachment/locking means 9 may include one or more, or combinations of belts, clamps, anchors, buckles, touch fasteners (e.g., Velcros), pegs, shackles, magnets, snap fasteners, suction pumps, adhesive strips, and the like.

Various materials, ingredients, chemical compounds, biological samples, devices, instruments, and tools needed in the task accomplishment may need to be kept separated before performing the task and interact only during the task performance. Said materials, ingredients, chemical compounds, biological samples, devices, instruments, and tools may be packed separately, individually or in groups, in small packages 10, cartridges, vials 11 with caps 12 and fastened in supports 13 to the bottom of the container, or in other suitable material containers that are included in the utilitarian-container, where the separate packaging prevents their contact, reaction, or inter-contamination before the use. Said materials, chemical compounds, biological samples, devices, instruments, and tools may be released by the user at the opportune moment during the task performance. The utilitarian-container may include pockets which may hang or may be affixed on the interior of the walls of the enclosure to better organize the materials, tools, devices and instruments in the enclosure. Prior to deployment and use, the utilitarian task-based container may be packed and sealed in a deflated state and the enclosure of the utilitarian container may include/contain all or part of the required materials, devices, tools and instruments needed for a specific task (e.g., experiment, test, procedure). Freeing the materials, tools, devices and instruments from their packages during the task accomplishment may be done in various ways, for example by ripping, cutting, or puncturing with a small tool also included in the utilitarian container.

The utilitarian-container may be used as an experiment container and may contain one or more of the following: test kits, microscopes, tools, equipment, a series of labware recipients comprising small beakers, Erlenmeyer flasks, laboratory flasks, test-tubes without or with seal cap, pre-labeled test-tubes, graduated cylinders and measuring cups, Styrofoam cups, culture tubes, pipets, graded pipets, aspirating pipets, petri dishes 14, jars, and the like, as required by the experiment. The utilitarian-container may further include/contain one or more tools and devices, such as: rounded tip scissors, spatulas, glass rods, small test tube racks, tongs, small filter funnels, filter paper, test tube holders, clamps, test tube clamps, thermo-isolating ceramic plates, thermo-conductive plates, textile pieces, optical prisms and prism kits, piezoelectric actuators to produce sparks as initiators of reactions, small batteries and heating elements, sensors 15, electric and electronic devices and instruments, and devices for illumination the experiment. Also, the utilitarian container may contain auxiliary materials such as adhesive tapes and small glue tubes, according to the needs of the experiments.

Some of the labware may be attached to the bottom of the container, or to its walls. Some of the labware and tools may be suspended from the upper side of the container with strips, small chains or other similar means, such that they are easy to manipulate during the operations/procedures required by the task.

The materials, samples, vials, cartridges and/or the casing in which they are packed may be numbered or coded by colors to, for example, signal the order of using them.

In an exemplary embodiment, the utilitarian-container is a single-use container which is discarded after the task was performed. In an exemplary embodiment, the utilitarian-container may be configured to be reusable, with or without deflation and re-inflation, such as when the performed procedures do not involve dangerous compounds released in the utilitarian-container. For utilitarian-containers configured to be reused, the container may include means for deflation and re-inflation, such as: controlled gas valves and taps, supplementary kits for gas generation, supplementary pressurized gas cartridges included in the container, external pumps with means to insert the re-inflation nozzle through the enclosure wall, and means of re-sealing the wall after re-inflation. Various ways of reclosing a plastic container/enclosure are known and may be used.

The utilitarian-container is configured to include and use gas or gas mixture which are compatible with the task/procedure to be performed. For example, for some tasks the use of nitrogen or inert gases may be required while a carbon dioxide atmosphere may not be used when combustion would need to be demonstrated in an experiment.

The user may manipulate the materials, devices, and instruments in the enclosure by using the provided gloves, fingers and inward protuberances. The utilitarian-container may include additional devices specifically designed for the purpose of the utilitarian container. For example, the utilitarian-container may include test tube holders, clamps, or glass rods configured to be used from outside the container and accommodated by the inward finger-like protuberances or other specially designed protuberances of the container.

The utilitarian-container may be configured to be securely disposed of after the experiments, tasks and/or procedures are performed. For example, the noxious gases may be released under a fume vent; the liquids inside the enclosure may be securely collected for recycling; and the containers, devices and tools may be recycled.

The utilitarian-containers may protect users by ensuring that their skin and body is not contaminated with material used during or resulting from the task procedures or experiments and by preventing all tools, parts and materials used in the task to reach the eyes, skin, mouth, or nose of the user. The utilitarian-containers may protect the juvenile users by preventing their skin and body contamination with any material used in or resulting during the task procedure or experiments. The utilitarian-containers may protect the juvenile users by preventing them from eating or drinking any element resulted from the experiment. The utilitarian-containers may protect users (e.g., juveniles) by preventing tools, parts and materials used in the task to reach the eyes, skin, mouth, or nose of the user.

The utilitarian container may include one or more pre-packed noxious gas absorbers, such as: absorbers based on porous charcoal or nanoparticles; absorbers and sponges used for collecting noxious or inconvenient liquids generated or used during the experiment; and chemical neutralizers.

The walls of the utilitarian-container may include outward protuberances formed as handles and grips that, when the container is inflated, allow its easy manipulation. The container may also include one or several attachment means, such as external belts, strips, rings, suction devices attached to its walls, which, alone or in conjunction with the grips and handlers, allow the securing of the container on the ground or on larger objects such as laboratory tables and furniture. The utilitarian-container may further include in its walls one or several permanent magnets. One or several movable permanent magnets may be placed in the container and may be used for fastening the container on metallic tables or objects with iron elements.

The technology is currently ripe for the conceiving, designing, and manufacturing at an affordable cost of large numbers of prepackaged kits to help medical and military personnel during in-the-field operations. The technology is currently ripe for the conceiving, designing, and manufacturing at an affordable cost of large numbers of prepackaged experiments and educational toys teaching elementary experimental and scientific knowledge.

The technology of prepackaged goods in airtight bags is well known and mastered. Manufacturing chemically resistant, electrically non-conductive, airtight containers of various plastic materials and a wide range of shapes and dimensions is also well mastered and may be employed by the utilitarian task-based container.

Also, the technologies for autonomously inflating a container by means of a chemical reaction between two or several compounds included in a container, by pressurized gas cartridges, or the like, or by external air or gas pumps or by pressurized canisters are well mastered and may be employed by the utilitarian task-based container. Means to prevent the overpressure in a container by using pressure control, pump control, overpressure valves, and external recipients for collecting noxious gases ejected from over-pressured recipients are also known and may be employed by the utilitarian task-based container. Devices, tools, and instruments for measuring and controlling various experimental parameters, conditions and results are also well known and may be employed by the utilitarian task-based container.

### Description of Several Preferred Embodiments

In exemplary embodiments of the invention, the inflated enclosure of the utilitarian-container may have a shape which is partially approximated by geometrical figures/shapes such as: a rounded parallelepiped shape; a barrel shape; a cylinder shape; a sphere shape; an ovoidal shape; an ellipsoidal shape; a super-ellipsoidal defined by:

$$\\ {( \frac{x}{a} )}^{2 n} + {( \frac{y}{b} )}^{2 n} + {( \frac{z}{c} )}^{2 n} = {d}^{{}^{} 2 n}$$

where n is an integer and a, b, c, d are positive real numbers. In other exemplary embodiments, the inflated-enclosure may have a shape including a first-part which is approximated by the above geometrical figures (e.g., the ovoidal, ellipsoidal, cylinder, sphere, barrel or super-ellipsoidal) and a second-part which is essentially a planar surface on which the first-part of the inflated enclosure lays. In yet other exemplary embodiments, the inflated container may have a shape which is partially approximated by geometrical figures/shapes such as: truncated cone; truncated pyramid; and truncated prism with essentially polygonal basis, such as quadrilateral, pentagonal, or hexagonal basis.

The utilitarian-container may be configured such that its dimensions depend on the enclosed task kit, the number of intended users, the amplitude of the movements required to accomplish the task, and the age and grade of the intended juvenile users in the case the experiment container is intended for children. Typical dimensions for parallelipipedic containers intended for a single user manipulating the container and actually performing the experiment may be 20 cm×20 cm×20 cm when no gloves are included, or 40 cm×30 cm×30 cm when the intended user is a child, or 60 cm×50 cm×40 cm, or any intermediate dimension. Larger and more complex tasks, or tasks intended for several users may require containers with the horizontal bottom surface similar to that of the typical laboratory table for one person, with heights between 25 cm and 60 cm. When the shape of the experiment container is essentially truncated (frustrated) ovoid, ellipsoid, super-ellipsoid, the parameters a, b and c may typically range between 10 cm and 40 cm. The skilled artisan will recognize that numerous variations of the shape and of the dimensions of the container may be used and may adapt the dimensions and shape of the utilitarian container depending on the task and purpose of the container.

The number of gloves with sleeves and the number of fingers and protuberances for tools and instruments as well as their dimensions also depend on the task and the intended number of users. Simple tasks for a single person may require only two gloves, while tasks requiring manipulations by at least two users may require six or eight gloves such that the users may employ any pair of gloves at their choice for reaching the desired region in the container. The gloves for larger containers or for tasks requiring ample movements may have long sleeves such that the hands of the users can reach to longer distances inside the container.

The enclosure may include one or more top and side view regions or panels of transparent enclosure material including optically-clear plastic, such as polyvinyl chloride and/or thermoplastic polyurethane (TPU) (so as to permit the operators to view inside the enclosure), chemically stable polymeric foil, or of composite foils. The polymeric foils may include those made of polystyrene, polypropylene, polyester (PET), ethylene vinyl acetate, or polyethylene terephthalate (PET). Multilayer laminates comprising several foils of the above or other suitable laminates may be used when a combination of properties is desired and cannot be obtained using single layers. In an exemplary embodiment the transparent enclosure material may be a thermoplastic polyurethane (TPU) of about 2 mil, or 4 mil, or 6 mil, or 8 mil, or 10 mil, or 12 mil thickness, or higher or other values as may be appropriate from a manufacturability, ease of use, visibility, flexibility, or other desirable material properties known in the art. The transparent enclosure material may be configured to have one or more of the following qualities: good resilience; abrasion resistance; hydrolytic stability and resistance to attack by microorganisms; durability (for puncture, tear resistance); clarity (for optimal viewing); water an oil impermeability; airtight and gas tight material; flexibility; chemically stability and stickiness. In an exemplary embodiment, the enclosure may be essentially entirely made of the above materials.

The walls of the utilitarian-container (e.g., enclosure 1) may be traversed through by tools such as pincers or stirring rods 7 that can be manipulated from outside the container walls. The walls of the utilitarian-container (e.g., enclosure 1) may have attached to their inner side (especially to the inner side of the fingers or gloves as already said) objects, tools and devices such as small vials, switchers, rods, cutters, and pincers.

The utilitarian-container may have the lowermost side reinforced (e.g., reinforced bottom panel). The utilitarian-container may include a variety of sensors 15 and devices, such as heaters for mild heating, inductances for contactless power supply, coils for creating rotating magnetic fields for magnetic stirrers, and cables for connecting electric devices inside the container with external devices. The reinforced bottom panel may be contiguous with the enclosure 1 and/or may be part of the enclosure 1. The reinforced bottom panel may be made of a different material than the rest of the enclosure and may be heat resistant, corrosion resistant, and/or may have other properties necessary for the intended task.

The utilitarian-container may include one or more of the following components: microscopes, spectrometers, magnifying glasses, diffraction gratings, light polarizing films, and systems of optical lenses which could facilitate testing and observation of experiments. The components referred above may be included and/or incorporated in the walls of the utilitarian-container.

The utilitarian-container may include pre-packed noxious gas absorbers and neutralizers, such as activated carbon-based gas absorber, absorbers and neutralizers for noxious or inconvenient liquids used or produced during the experiment, and biological neutralizers. The utilitarian-container may include pre-packed desiccants to absorb moisture.

The utilitarian-container may include one or more supplementary-airtight-containers connected to the enclosure 1 (e.g., via one or more safety valves) and configured to receive gas from the enclosure (e.g., in case the pressure inside the enclosure becomes larger than a threshold pressure). The utilitarian-container may include one or more safety valves configured to discharge gas in the one or more supplementary-airtight-containers. The supplementary-airtight-containers may have large volume so as to accommodate extra materials and gases generated inside enclosure 1. The supplementary-airtight-containers are airtightly sealed to the experiment enclosure 1.

The utilitarian-container may have the walls traversed by electric cables for power supply, without compromising the sealing of the container, through openings sealed with gaskets or through other means known to those skilled in the art.

The walls of the utilitarian-container may include sheets or layers with conductive properties when the experiment requires a Faraday cage-like container, or may have magnetic properties, or a combination of them. The technology for manufacturing such materials and walls is well known, as for example the technology of electrically conductive polymer sheets (conductive elastomer).

The utilitarian-container may include one or more displays and indicators for displaying the results of measurements performed on the utilitarian-container (e.g., via sensors and detectors) and the processes taking place inside the container. For example, the displays may display the temperatures/pressures inside the enclosure, test results, sensor measurements, parameters associated with the experiments and tests. The displays may be placed inside the enclosure, on the walls of the enclosure, and/or as flexible displays incorporated into or attached to the enclosure.

The utilitarian-container may include, or it may carry in its or on its walls, differential pressure sensors that connect to an alarm circuit to signal when the inside pressure is too low or too high for safely using the utilitarian container. The alarm may be generated locally, or, when connected to communication means, the alarm may be generated at the supervisor terminal, over the Internet, or a combination of them.

The enclosure or the walls of the utilitarian-container may include at least one high transparency portion forming a window for taking pictures and/or videos via one or more communication means such as cameras 16. The utilitarian-container may further include means for connecting and/or remote controlling the cameras 16 (e.g., via communication means such as Wi-Fi) with one or more computers, a network, or the internet. The cameras 16 may be configured to acquire videos and images of the experiments and tests performed inside the container, to transmit the images and videos to the computers (e.g., via a network or the internet), and to display said images and videos at the computers. This way other people (e.g., teachers, supervisors, instructors) are able to view (e.g., remotely) and to monitor (e.g., in real-time) the experiments, test and procedures performed inside the enclosure and to provide advice and guidance in real-time regarding the tasks to be performed. In an exemplary embodiment, the cameras may act as IoT devices and may connect remotely to people who can help with the task performance or who monitor the work for evaluation purposes. The recordings and communication means may be used remotely for helping during the learning process and/or may help with correcting and self-correcting wrong and/or inefficient movements of the operator inside the container.

The tools used in conjunction with or included in the utilitarian-container may have embedded position and inertial sensors/modules, such as the ones based on MEMS (micro-electromechanical systems). For example, the utilitarian-container may include position and inertial modules including 3D accelerometers and 3D gyroscopes. The measurements performed by the position and inertial sensors may be combined with each other and with the measurement performed by other sensors for other variables (e.g., temperature, pressure).

The utilitarian-container may include and employ other type of sensors, such as capacitive, inductive, ultrasound-based, or time of flight-based sensors. Such sensors may be used instead or in conjunction with inertial sensors to acquire information regarding the position and movement of hands and tools during task performance. Such position and motion sensors may be placed on one or more of the following: tools, gloves, operators' hands or wrists, on mobile parts inside the container, on the walls of the container.

The information received from the position and inertial sensors may be used to determine instant and the evolution in time of 3-dimensional (3D) positions, speeds, accelerations, and angular displacements (gyroscopic). The acquired data may be processed and machine learning (ML) may be performed (on chip or remotely) to analyze the information. The information received from the position and inertial sensors may be used in conjunction with machine learning (ML) algorithms and with visual observations to perform a detailed analysis of the operators actions and skills, to detect movement errors, and to provide feedback regarding operators performance. The feedback may be received from ML system and/or from a human instructor. The feedback may be used to improve learning.

Especially for utilitarian-containers designed for training and education purposes, the use of tools such as pincers already attached to the fingers 4 have the advantage of indicating what are the best positions of the experimenter's hand 17 and fingers for efficient manipulation and may teach the students the right movements by placing constraints to movements. The placements of gloves and fingers in the utilitarian-container, the general configuration of the enclosure, the video acquisition by camera, the information provided by various sensors (e.g., position and inertial sensors), and the ML algorithms are all configured to work together so as to provide an efficient educational process leading to students acquiring the desired skills.

The embodiments presented in this disclosure merely serve as exemplary embodiments of the utilitarian-container and it will be apparent to those skilled in the art that various modifications and variations can be made in the present invention without departing from the spirit or scope of the invention. The utilitarian-container disclosed herein may be adapted for various circumstances, uses, experiments, tasks, and processes (e.g., in-the-field medical interventions, military missions, education, professional tasks) without departing from the spirit or scope of the invention.

Several examples of using the utilitarian-container to perform educational experiments are provided in the following.

In a first exemplary embodiment, the utilitarian-container may be used to perform a chemistry experiment. Materials and tools needed for the experiment may come prepacked in the enclosure of the utilitarian-container while in uninflated state. The experiment to be performed relates to chemical reactions between stronger acids, such as sulfuric acid and hydrochloric acid, and metals, such as iron (Fe) and zinc (Zn), according to the redox reaction Metal+acid→hydrogen+salt, specifically Zn+H₂SO₄→H₂+ZnSO₄.

In the redox (oxidation-reduction) reaction: oxidation: Zn⁰−2e⁻→Zn^{II} is followed by reduction: 2H^{I}+2e⁻→2H⁰, where the oxidizing agent is H₂SO₄, with Zn the reducing agent, while the resulting salt is zinc sulfate, ZnSO₄. The salt crystalizes as a white powder. The hydrated zinc sulfate (ZnSO4·7H2O) also crystallizes and in nature it forms the mineral Goslarite. The salt is used in agriculture (e.g., as a fertilizer and in small amounts in animal food) and in toothpaste.

In this embodiment, the enclosure of the utilitarian container includes a clear plastic test tube (5 to 15 ml) with tight cap, containing a few mg of diluted sulfuric acid, a small container of rough powder or granules of zinc hermetically sealed (zinc is pyrophoric, with strong oxidation in air and with a tendency to burn), and a test tube clamp. For this experiment, the container should be inflated with air or with a gas mixture that includes oxygen, for the produced hydrogen to burn. The test tube may be made of polystyrene that has a good resistance to sulfuric acid up to 35% concentration. Polypropylene tubes are not suitable at concentrations higher than 10% of dilute sulfuric acid. Because the zinc sulfate is hygroscopic and soluble in water, the container preferably includes a small permeable package of desiccant. Because dangerous gases are produced, the walls of the utilitarian-container may be reinforced. The utilitarian-container may include a relief safety vane opening in a normally deflated secondary chamber of the container that has elastic walls and is able to receive the gas from the main enclosure thus reducing pressure to a safe level that cannot be exceeded by the gas released from the materials included in the container. The size and wall strength of this secondary chamber is calculated based on the task, for instance in this case the specific quantities of experimental chemical compounds and the order in which they are mixed to accomplish the educational experiment task.

In the experiment, the students are able to watch the “dissolving” effect of the acid on the metal and the bubbling of the hydrogen. In a more detailed experiment, the container may comprise a small piezoelectric spark generator or lighter used to put on fire at the opening of the test tube of the small amount of hydrogen produced during the reaction. Also, the container may comprise a hot plate or a small evaporating basin for crystallization of the salt, and an intelligent pressure sensor with a suitable display inside the container to determine any change in pressure due to the hydrogen produced by the reaction, when the hydrogen is not burned. When the hydrogen is not burned, determining the pressure change in the container with the pressure sensor, the student can derive the quantity of hydrogen generated using the gas laws. When the hydrogen is burned, the reaction consumes part of the oxygen in the container and the pressure drops, with partial deflation of the container.

The experiment may include a third part, where the container includes a capsule for the release of CO2. The student repeats the redox reaction using a second set of sulfuric acid and zinc materials. Then the student tries to set alight the hydrogen produced; because the oxygen is missing, there will be no fire, proving that the hydrogen reacted with oxygen, not with other element in the container atmosphere. An experiment description sheet and a student instruction sheet may be included in the experiment package with the container.

In a second exemplary embodiment, the utilitarian-container may be used to perform a displacement reaction. The container may comprise a clear plastic test tube (5 to 15 ml) with tight cap, containing a few mg of diluted sulfuric acid, a small, sealed bag of rough copper powder or granules, a small zinc strip, a test tube clamp, and a pincer for handling the zinc strip. The tube clamp and the pincer may be external to the container and may be used for manipulation inside the container through specially designed inward finger-type protrusions. In the experiment, the reaction of copper with the sulfuric acid is performed, as in the first example above. After that the zinc strip is introduced in the solution of copper sulfur salt. The student watches the deposition of copper over the zinc strip during the displacement reaction, where the zinc extracts the copper and replaces it in the new sulfuric salt formed. An experiment description sheet and a student instruction sheet may be included in the experiment and may be pre-packaged with the container.

In a third exemplary embodiment, the utilitarian-container comprises a small piece of nylon-based textile, a small plate of polypropylene or vinyl (PVC), two small strips of aluminum (Al) foil, a small tube of adhesive, and a small package of fine powder. The student first folds the small strip of Al along its median line and glues it at the median line on the plate, toward one end of the plate. Then, the student rubs the plate with the textile on the side opposed to the Al foil, without touching the Al foil, while observing it. Subsequently, the student pours the powder on the plate and agitates the plate to create a more uniform coverage. Then, the student reverses the plate such that the powder falls to the bottom of the container (student flips the plate upside-down), except some powder remains sticking to the plate due to electrostatic charging. The student observes the distribution of the powder on the plate, according to the electric charge distribution created by tribocharging. For this experiment, the container preferably has conductive walls for preventing electrostatic charging of the walls, charge transfer to the operator, and electrostatic attraction of the powder for visualizing the electrostatic charging by the walls.

In a fourth exemplary embodiment, the utilitarian-container may be used for food preparation. The initially deflated container is a vacuumed container including several or all ingredients for the partial preparation of a meal, such as raw meat and vegetables that may produce unpleasant odors, may be unhygienic or messy to manipulate, or otherwise unpleasant to use and openly or bare-handedly prepare. The ingredients may be separately prepacked such that they do not to mix together before use and such that the user can determine the mixing proportions at will, in a personalized manner. The said container includes means for self-inflation, for example a compressed gas cartridge, where these means are controllable from outside the container for the gas release. Alternatively, the container has means for secure connection with an external pump for inflation. The container may include tools for the food preparation, such as small cups, dishes, aluminum foil, measuring spoons, small plastic knives, or various gauges for food. After the user prepares the desired food composition according to taste, using fully or partially the ingredients, the bag is cut and the unused remains are discarded within the container in a hygienic manner or are preserved for further use, while the prepared composition is used for the final stages of food preparation, using a stove, oven or a similar coking appliance.

The means for self-inflation may have a safety feature consisting in a mechanism of automatic gas release after a specified time corresponding to the ingredients' degradation time to signal that the contained food is unhealthy. The automatic gas release mechanism may be based on an electronic timer or may be based on a pressured gas cartridge with a gas release mechanism including a degradable stopper of the gas vane. The degradable stopper may be biodegradable and release the contents after the food in the container has spoiled to indicate to the user the food is unsafe to eat.

The utilitarian task-based container described in this application address challenges associated with in-the-field and outdoors preparation of materials and tools for military, medical and other applications. The utilitarian-container is configured to be used for preparing materials and tools outdoors, such as during in-the-field military missions and during in-the-field medical interventions. In-the-field operations may include medical interventions on inhabitants of remote regions, during rescue operations in wilderness, and in environments which lack the sterility of a hospital operating rooms or of laboratories (e.g., tents, cottages, residential rooms, non-operating rooms in hospitals, etc.). The utilitarian-container may include batteries configured to provide power to the devices which may be needed during the tasks and procedures.

The utilitarian task-based container described in this application also address the challenges associated with safely and efficiently performing science experiments in educational environments and when the users are children.

The utilitarian task-based container allows users to prepare materials and tools in a relatively sterile environment (e.g., as sterile as feasible under the conditions) by preventing contaminants from the outer environment (i.e. outside of the enclosure) to reach the inner of the enclosure where materials and tools are prepared. The utilitarian-container is configured to ensure that substances, dust, and contaminants from the outer environment (outside the enclosure) are not reaching the inside of the enclosure which is the site where materials and tools are prepared. The utilitarian-container may be configured to ensure that substances, dust, and contaminants from inside the enclosure are not reaching users' bodies. The utilitarian-container provides a barrier protecting operators from exposure to contaminants present or generated inside the enclosure.

Prior to use, the utilitarian-container is configured to be packed into a portable bag (e.g. backpack) so as to be easy to carry in the field. While packed, the surgical enclosure may be folded like a surgical gown. The utilitarian-container may be configured to be light, ergonomic and easy to deploy.

The utilitarian-container may be configured to be single use (i.e. after use it will be discarded). The utilitarian-container may be configured to be used multiple times and for multiple experiments, tests or procedures.

Embodiments of the invention are described herein with reference to figures and illustrations that are schematic illustrations of idealized embodiments (and intermediate structures) of the invention. As such, variations from the shapes of the illustrations as a result, for example, of manufacturing techniques and/or tolerances, are to be expected. Thus, embodiments of the invention should not be construed as limited to the particular shapes of regions illustrated herein but are to include deviations in shapes that result, for example, from manufacturing.

The aspects of the invention in this application are not limited to the disclosed operations and sequence of operations. For instance, operations may be performed by various elements and components, may be consolidated, may be omitted, and may be altered without departing from the spirit and scope of the present invention.

The portable surgical systems disclosed herein may include alternate or additional sections which could be added based on procedural needs, such as to accommodate additional instruments or users. The above embodiments presented in this disclosure merely serve as exemplary embodiments and it will be apparent to those skilled in the art that various modifications and variations can be made in the present invention without departing from the spirit or scope of the invention.

The terminology used herein is for the purpose of describing particular embodiments only and is not intended to be limiting of the present disclosure. The inventions herein may be embodied in many different forms and should not be construed as limited to the exemplary embodiments set forth herein. Rather, these exemplary embodiments are provided so that this disclosure is thorough, and will fully convey the scope of the invention to skilled artisans.

1. An utilitarian-container enabling users to perform specific-tasks, the utilitarian-container comprising:
(a). an inflatable, flexible, and fluids-impermeable enclosure;
(b). an experiment-kit comprising a plurality of inner-elements disposed inside the enclosure,
wherein the inner-elements further comprise materials, devices, and tools for performing the specific-tasks;
wherein the utilitarian-container comes in at least the following configurations: a deflated-state, an operational-state, and a discard-state;
wherein, in the deflated-state and the operational-state, the configurations and positions of the inner-elements inside the enclosure are pre-arranged such as to enable users to efficiently perform the specific-tasks;
wherein the enclosure has at least part of its walls transparent thereby enabling users to see inside the enclosure;
wherein the enclosure comprises one or more ports enabling users to access and handle, from outside the enclosure, the inner-elements inside the enclosure while in the operational-state;
wherein the enclosure comprises locking-means for attaching or affixing the enclosure to the ground, or to a table, or to affixed objects;
wherein the enclosure comprises inflation-means for inflating the enclosure.

2. The utilitarian-container of claim 1, wherein the enclosure comprises self-inflating ribs or girds configured to reinforce the enclosure by supporting the walls of the enclosure, wherein the ribs or girds are attached to said walls inside or outside the enclosure.

3. The utilitarian-container of claim 2, wherein the ribs and girds are configured to be inflated at a high pressure so as to acquire relative rigidity, the ribs and girds are airtight, and wherein the ribs and girds comprise means for autonomous inflation or means for inflation from an external pump.

4. The utilitarian-container of claim 1, wherein the enclosure comprises one or more of the following: inward protuberances; gloves; and sleeves attached to the enclosure at positions that are convenient for performing the intended task;
wherein the positions, dimensions and materials of the protuberances, gloves, and sleeves are configured to fit the circumstances of the prospective users and of the specific-tasks to be performed;
wherein circumstances of the prospective users comprise one or more of the following: the age of the intended users, the hand and finger size of the prospective users, and the physical and mental abilities of the prospective users;
wherein the specific-tasks to be performed comprise one or more of the following:
preparing materials, preparing tools, and preparing equipment for in-the-field medical interventions;
performing medical training and/or medical tests;
preparing materials, tools and equipment for military in-the-field operations and missions;
performing in-the-field tests or experiments;
performing scientific and technical experiments for training or educating juveniles and young students.

5. The utilitarian-container of claim 4, wherein one or more of the inward protuberances are shaped as fingers disposed such that the user can pinch, squeeze or otherwise manipulate objects or materials inside the enclosure.

6. The utilitarian-container of claim 4, wherein one or more inward protuberances are shaped according to the shape of specific tools or instruments that the users need to manipulate during the task accomplishment, and wherein the specific tools are pre-attached to the protuberances.

7. The utilitarian-container of claim 1, wherein the inflation-means comprise one or more of the following: compressed gas cartridges and packed chemicals that, when put in contact, generate the required volume of gas for the enclosure inflation; wherein the inflation-means are autonomous self-inflation means.

8. The utilitarian-container of claim 1, wherein the enclosure comprises one or more magnifying glasses or systems of glasses to allow better observation of the inside of the enclosure.

9. The utilitarian-container of claim 1, wherein the walls of the enclosure comprise one or more regions that allow the airtight insertion through the enclosure of needles or other thin devices and tools, or a combination of them, while the wall recovers its airtight property after the inserted device is removed.

10. The utilitarian-container of claim 1, wherein the enclosure walls are configured to be impermeable, resistant, and inert to materials and substances inside the enclosure; and
wherein the said materials and substances comprise one or more of the following: materials and chemicals used or generated during testing, experiments and procedures performed during the specific-tasks; materials generated by chemical reactions; chemical acids; chemical bases; chemical noxious compounds; noxious gases; toxic materials or materials that produce toxic vapors or liquids; materials that produce noxious particles; nano-materials that are hazardous to the human body; biological materials or microorganisms; and materials or compounds that are noxious or inconvenient for direct contact or open-atmosphere manipulation.

11. The utilitarian-container of claim 1, wherein the inner-elements comprise one or more of the following: packages and casings holding materials or tools, samples, vials, cartridges, devices; wherein the inner-elements are numbered or color coded to signal their order of use.

12. The utilitarian-container of claim 1, further comprising one or more of the following elements configured to enable task performance: electrical devices and equipment; electronic devices; magnetic materials; electronic circuits.

13. The utilitarian-container of claim 1, further comprising one or more of the following elements configured to enable task performance: measurement devices, sensors, signal processing devices, process control devices, data communication means, and computing devices.

14. The utilitarian-container of claim 1, further comprising one or more of the following elements configured to enable task performance: devices and tools for manipulating materials; squeezers; dispensers; sticks for mixing; electrical mixers; small heating or cooling elements; and receptacles of various forms.

15. The utilitarian-container of claim 1, further comprising one or more of the following elements configured to enable task performance: noxious gas absorbers, absorbers for noxious or undesired fluids used or generated during task performance, chemical or biological neutralizers and absorbers.

16. The utilitarian-container of claim 1, further comprising:
(a). one or more safety-valves configured to discharge gas from inside the enclosure;
(b). one or more supplementary-containers connected to the enclosure via the safety-valves, the supplementary-containers being configured to receive from the enclosure excess gases and materials;
wherein the supplementary containers are airtightly sealed to the enclosure around the safety-valves and the supplementary containers have volumes configured contain at a safe pressure excess gases generated during the task accomplishment;
wherein the supplementary-containers are airtight and flexible; and
wherein the supplementary-containers are in an uninflated state prior to the safety-valves discharging gases into the supplementary-containers.

17. The utilitarian-container of claim 1, further comprising a differential pressure sensor connected to an alarm configured to generate an alarm-signal when the inside pressure is too low or too high for safe use, wherein the alarm may be generated locally, at a supervisor terminal, and/or over the internet.

18. The utilitarian-container of claim 1, further comprising cable-ports configured to accommodate one or more power supply cables and signal carrying cables securely and air-tightly traversing the enclosure wall through the cable-ports.

19. The utilitarian-container of claim 1, further comprising one or more displays, wherein the displays are placed inside the container, embedded in the walls of the container, or placed on the walls in the form of a flexible display,
wherein the displays show one or more of the following: measured process parameters, process control values, information for the user, images and videos from inside the enclosure.

20. The utilitarian-container of claim 1, further comprising one or more of the following:
outward protuberances, disposed on the wall of the enclosure, formed as handles and grips for manipulation of the utilitarian-container by the users, and
one or several attachment-means configured to secure of the enclosure on the ground or on larger objects, wherein the attachment-means comprise one or more of the following: belts, strips, suction cups, other suction devices that.

21. The utilitarian-container of claim 1, wherein the container is reusable and includes kits of materials and receptacles for several successively performed tasks or for task repetitions; wherein the container is configured to be reusable with or without deflation and re-inflation.

22. The utilitarian-container of claim 1, wherein the enclosure is configured to be deflated and re-inflated, and wherein the utilitarian-container further comprises:
means for controlled deflation comprising controlled gas valves, taps, and other deflation-means; and
means for controlled re-inflation comprising kits for gas generation, pressurized cartridges, external pumps, other rei-inflation means.

23. The utilitarian-container of claim 1, wherein the container is reusable and comprises:
one or more airtight ports allowing for the removal of consumed materials and tools and for the replacement with other materials and tools for further use,
wherein the airtight ports have means for resealing comprising one or more of airtight zips, re-sealable adhesive tapes, other resealing means.

24. The utilitarian-container of claim 1, wherein the enclosure walls are configured to provide electrical shielding and to perform as a Faraday cage, and wherein the walls comprise one or more of the following: conductive polymers, thin transparent metallization films, thin transparent conductive films and foils.

25. The utilitarian-container of claim 1, wherein the enclosure walls are configured to provide electrical shielding and to perform as a Faraday cage, and wherein the walls comprise one or more of the following: a wireframe mesh, a transparent, thin conductive wireframe deposited on the walls' surface, composite portions comprising conductive material.

26. The utilitarian-container of claim 1, wherein the enclosure walls comprise magnetic materials configured to be used in magnetism experiments or configured to provide electromagnetic shielding.

27. The utilitarian-container of claim 1, further comprising one or more magnets configured to allow the user to firmly attach the enclosure to tables or other external objects that are made of or include in their structure iron parts.

28. The utilitarian-container of claim 1, further comprising locking-means for attaching the enclosure to the ground or surroundings, the locking-means comprising one or more of the following: belts, clamps, anchors, buckles, touch fasteners (Velcros), pegs, shackles, magnets, snap fasteners, suction pumps, adhesive strips, and the like.

29. The utilitarian-container of claim 1, further comprising visual indicators for overpressure.

30. The utilitarian-container of claim 1, further comprising sensors configured to detect corrosion or otherwise degradation of the enclosure wall.

31. The utilitarian-container of claim 1, further comprising cleaning-means configured to remove liquid and particulate residues from inside the enclosure, the cleaning-means comprising one or more of the following: sponges; fabric; dust cloths; magnet-operated sponges or fabric.

32. The utilitarian-container of claim 1, further comprising light sources configured to light the inside of the enclosure.

33. The utilitarian-container of claim 1, wherein the enclosure walls comprise a dark light reducing part or side configured to absorb bright uncomfortable light inside the enclosure.

34. The utilitarian-container of claim 1, further comprising a built-in safety mechanism activating a gas-release-vane after a trigger-time corresponding to the ingredients degradation time;
wherein the safety mechanism is configured to signal that the contained materials are unusable or dangerous;
wherein the safety mechanism comprises an electronic timer or a device configured to detect degradation of materials.

35. The utilitarian-container of claim 1, further comprising one or more optical-components configured to allow for better viewing the inside of the enclosure and for performing optical experiments, the optical-components comprising one or more of the following: magnifying glasses; optical lenses; polarizing films; diffraction gratings; lasers; light sources.

36. The utilitarian-container of claim 1, further comprising one or more of the following: inductances, antennas, and circuits configured to provide contactless power supply for powering the devices inside the container.

37. The utilitarian-container of claim 1, further comprising antennas and/or communication devices, wherein the antennas and the communication devices are incorporated into the enclosure's walls and/or are disposed on the walls.

38. The utilitarian-container of claim 1, further comprising one or more sensors incorporated into enclosure's walls.

39. The utilitarian-container of claim 1, further comprising materials or prepacked materials for neutralizing of the noxious compounds resulted from the experiment, including neutralizers for noxious gases and liquids.

40. The utilitarian-container of claim 1, wherein the bottom of the enclosure comprises one or more of the following: a structurally reinforced region, sensors, devices, heaters for mild heating, planar inductances and capacitances for experiments, coils for creating rotating magnetic fields for magnetic stirrers, and cables for connecting electric devices inside the container with external devices.

41. The utilitarian-container of claim 1, comprising:
tools and devices including one or more of the following: position-sensors, inertial-sensors, and cameras; and
data processing modules configured to acquire and process data from the tools and devices;
wherein the data processing module runs machine learning (ML) algorithms using the data from the sensors and cameras configured to create performance-parameters and to use said performance-parameters to perform one or more of the following: analyze user's skills, detect movement errors, and provide feedback and instructions to the user regarding user performance of the specific-tasks.

42. The utilitarian-container of claim 1, wherein in the deflated-state the utilitarian-container comes as a prepacked-kit configured to be deployed in the operational-state by inflating the enclosure.
